(19) United States
(12) Patent Application Publication
(10) Pub. No.: US 2019/0376106 A1
(43) Pub. Date: Dec. 12, 2019

(54) METHOD FOR ANALYSING A SPRAY GENERATED BY A DEVICE FOR DISPENSING FLUID PHARMACEUTICAL PRODUCT

(71) Applicant: APTAR FRANCE SAS, LE NEUBOURG (FR)

(72) Inventor: Julien BOISVILLIERS, Crosville La Vieille (FR)

(73) Assignee: APTAR FRANCE SAS, LE NEUBOURG (FR)

(21) Appl. No.: 16/477,300

(22) PCT Filed: Jan. 12, 2018
(86) PCT No.: PCT/FR2018/050074

§ 371 Date: Jul. 11, 2019

(30) Foreign Application Priority Data
Jan. 16, 2017 (FR) 1750318

(51) Int. Cl. C12Q 1/04; G01N 33/483; G01N 21/45; B05B 12/08

(52) U.S. Cl. CPC C12Q 1/04; B05B 12/084; G01N 21/455; G01N 33/483

(57) ABSTRACT

A method of analyzing a spray generated by a spray device for spraying pharmaceutical fluid, the method comprising the following steps:
providing a spray head (1) of a spray device for spraying pharmaceutical fluid, said spray head (1) including a spray orifice (2);
causing a test fluid to pass through said spray head (1) towards said spray orifice (2), said test fluid being air at a temperature that is different from ambient temperature;
displaying, by strioscopy, the flow of test fluid leaving said spray orifice (2); and
analyzing said display of the test-fluid flow so as to determine whether or not the test-fluid spray coming from said spray head complies with predetermined specifications.

The present invention relates to a dispenser, and to a method of analyzing a spray generated by a spray device for spraying a pharmaceutical fluid.

Spray devices for spraying pharmaceutical fluid are well known. In particular in nasal-spray applications, the therapeutic effectiveness of the sprayed fluid may depend on the properties of the spray generated while the device is being actuated. In known manner, at the end of the assembly line, i.e. once the spray device is assembled, and just prior to being sent to the pharmaceutical-fluid manufacturer for assembly onto a corresponding reservoir, a certain number of samples of assembled devices are laboratory tested so as to verify whether the properties of the spray correspond to pre-defined specifications.

A drawback of that system is that is relates to assembled devices, and thus destroys the devices which, after being tested, can no longer be delivered to the customer. Furthermore, the system requires human verification of the tested devices, and is thus not suitable for being completely automated.

The document “Assessment of fuel spraying using schlieren system” by Marek Klimkiewicz, Agriculture No. 65, Vol. 2015, pp. 119-126, describes the use of strioscopy to analyze fuel sprays coming from various fuel injectors.

An object of the present invention is to overcome the above-mentioned drawbacks.

In particular, an object of the present invention is to provide a device and a method of analyzing spray that do not destroy the tested devices.

Another object of the present invention is to provide a device and a method of analyzing spray that is largely automated.

Another object of the present invention is to provide a device and a method of analyzing spray that is simple and/or inexpensive to manufacture, to assemble, and to use.

The present invention thus provides a method of analyzing a spray generated by a spray device for spraying pharmaceutical fluid, the method comprising the following steps:
providing a spray head of a spray device for spraying pharmaceutical fluid, said spray head including a spray orifice;
causing a test fluid to pass through said spray head towards said spray orifice, said test fluid being air at a temperature that is different from ambient temperature;
displaying, by strioscopy, the flow of test fluid leaving said spray orifice; and
analyzing said display of the test-fluid flow so as to determine whether or not the test-fluid spray coming from said spray head complies with predetermined specifications.

Advantageously, said analyzing step includes determining the cone angle of the test-fluid spray.

Advantageously, said analyzing step includes an image-processing step for processing said display of the test-fluid flow.

Advantageously, said predetermined specifications include a predetermined spray cone angle, such that test-fluid sprays having a cone angle that is greater than or equal to said predetermined spray cone angle are classed as being compliant, and test-fluid sprays having a cone angle that is less than said predetermined spray cone angle are classed as being non-compliant.

The present invention also provides a device for analyzing a spray generated by a spray device for spraying pharmaceutical fluid, the device comprising:
a spray head of a spray device for spraying pharmaceutical fluid, said spray head including a spray orifice;
means for causing a test fluid to pass through said spray head towards said spray orifice, said test fluid being air at a temperature that is different from ambient temperature;
a strioscopic setup for displaying, by strioscopy, the flow of test fluid leaving said spray orifice; and
analyzer means for analyzing said display of the test-fluid flow so as to determine whether or not the test-fluid spray coming from said spray head complies with predetermined specifications.

Advantageously, said analyzer means include measuring means for measuring the cone angle of the test-fluid spray.

Advantageously, said analyzer means include image-processing means for processing said display of the test-fluid flow.

Advantageously, said strioscopic setup comprises a camera, an objective lens, a light source, generator means for generating a test-fluid flow, a display zone, at least one collimator lens, and a filter.

Advantageously, said strioscopic setup further comprises a concave and/or parabolic mirror.

Advantageously, said filter is a point, a wire, or a blade.

These characteristics and advantages and others appear more clearly from the following detailed description, given by way of non-limiting example, and with reference to the accompanying drawings, in which:

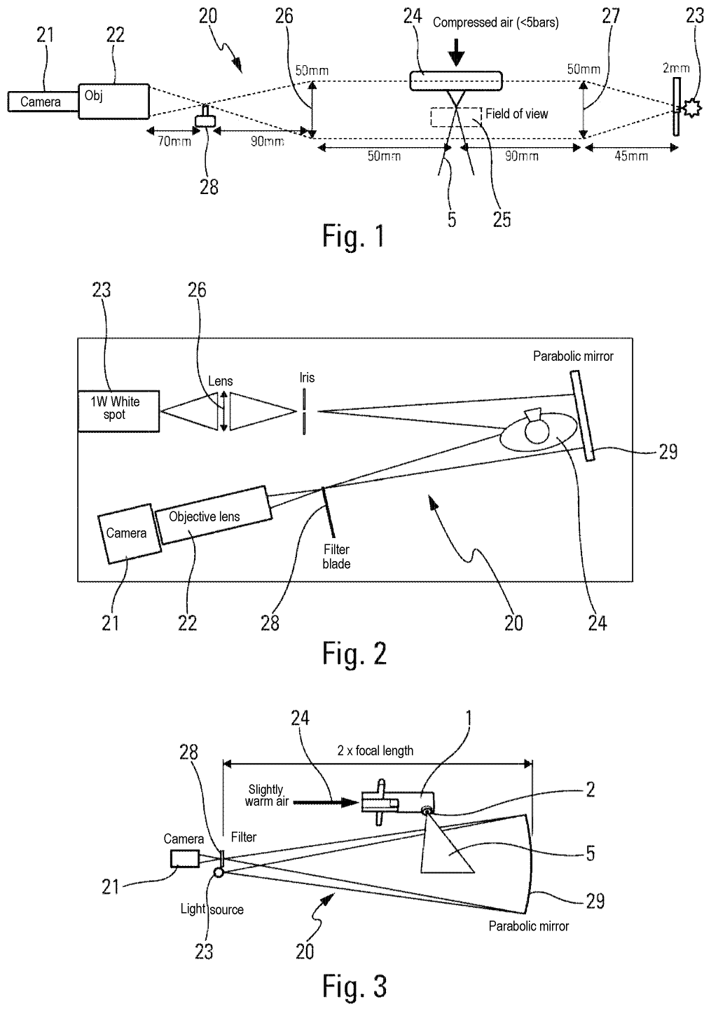

FIG. 1 is a diagrammatic view of a device for analyzing a spray, in a first advantageous embodiment;

FIG. 2 is a diagrammatic view of a device for analyzing a spray, in a second advantageous embodiment;

FIG. 3 is a diagrammatic view similar to the view in FIG. 2, of an advantageous variant embodiment;

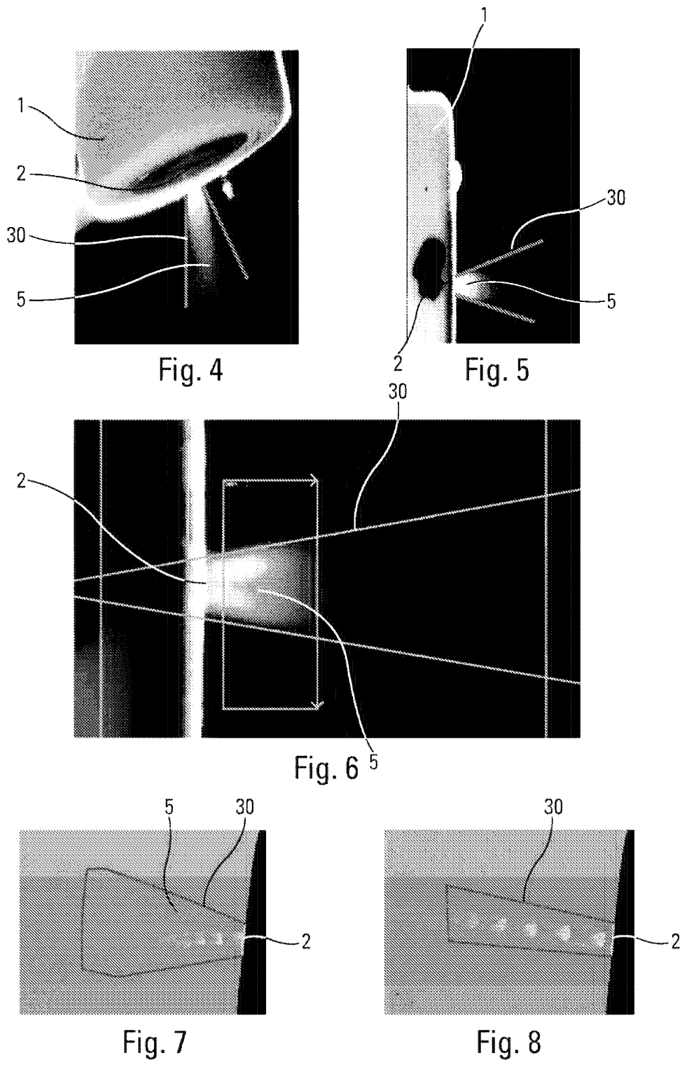

FIGS. 4 to 6 are diagrammatic views showing images obtained by a method of the invention;

FIG. 7 is a diagrammatic view of a compliant test-fluid spray obtained by a method of the invention; and

FIG. 8 is a view similar to the view in FIG. 7, showing a non-compliant test-fluid spray.

An object of the invention is to improve the quality of spray device inspection. To do this, the invention envisages using strioscopy to analyze in automatic manner the cone of the spray emitted by spray devices.

Strioscopy, or the schlieren method, is an optical display method that makes it possible to isolate in an image the details and small variations, in particular small variations in refractive index as happens during compression of air or other fluids. Graphically, the fundamental idea of the method is to remove light that has not been deflected by the object, e.g. the fluid under study. Specifically, only rays that have been deflected by said object correspond to turbulence or to optical high spatial frequencies. To achieve that, an image is made initially of the light source, preferably an incoherent light source, e.g. by means of a converging lens. Rays that have not been subjected to deflection (zero spatial frequencies) pass through the precise location of the geometrical image. These rays are eliminated with a filter. The other rays, those that have been deflected, are not focused at the same location and they can thus pass through in order to form a filtered image. In summary, the smooth background of the image is eliminated and consequently, the details or turbulence of the object, that were buried in the smooth background, become visible.

The filter used may merely be a point, a wire, or a blade, e.g. of the “Foucault knife-edge” type.

Strioscopy is an application of optical spatial Fourier filtering. Specifically, Fraunhofer diffraction indicates that a lens creates, in its image focal plane, the Fourier transform of the object in question. In this plane, it is thus possible to see the spatial frequencies associated with the object, and the filter is placed in the same plane in order to eliminate some of the spatial frequencies. This wave interpretation of strioscopy makes it comparable to high-pass filtering.

In the context of the present invention, the object is to show a flow of a test fluid, namely a jet of air at a temperature that is different from ambient temperature, e.g. heated air coming from the spray orifice 2 of a spray head 1 of a spray device for spraying pharmaceutical fluid, and to observe it by means of a camera. In a variant, it is also possible to use a jet of air at a temperature that is lower than ambient temperature.

FIG. 1 shows a strioscopic setup 20 in a first advantageous embodiment.

In this embodiment, a camera 21 associated with an objective lens 22 is arranged on one side of the setup 20, and a light source 23 is arranged on the opposite side. Generator means 24 for generating compressed air are provided so as to deliver a flow of air, preferably at a temperature that is different from ambient temperature, and pass it through a spray head 2 that is arranged in a display zone 25 that is arranged between two collimator lenses 26, 27. A blade or diaphragm 28 is provided in front of the objective lens 22 so as to interrupt the beam and thus filter the image, and thereby display the spray, as explained above.

The following components of standard type may be used for this setup:

| Components | Description |
|---|---|
| Camera | Manta 1292 × 964 @ 30 hertz (Hz) |
| Objective lens | 85 millimeters (mm) f: 1.8 |
| Collimator lens | 50 mm |
| Collimator lens | 50 mm |
| Lamp | White LED |
| Diaphragm | 2 mm |
| Pulse | Smartek 10 microseconds (μs), 12 amps (A) |

FIGS. 2 and 3 show two variants of another advantageous embodiment in which the strioscopic setup includes a concave and/or parabolic mirror 29. This embodiment is more compact, with the light source 23 and the camera 21 arranged on one side, and the mirror 29 arranged on the opposite side. The overall operation is similar to the setup in FIG. 1.

It should be observed that the images obtained for displaying the spray may comprise static images (photographs) and/or videos.

In order to analyze the displayed sprays, the invention includes analyzer means 30 for determining whether or not the test-fluid spray coming from said spray head complies with predetermined specifications.

In particular, the analyzer means 30 may include measuring means for measuring the cone angle of the test-fluid spray.

Optionally, image-processing means for processing the displays of the test-fluid spray may be used to perform said analysis.

Thus, the predetermined specifications may include a predetermined spray cone angle, such that sprays having a cone angle that is greater than or equal to said predetermined spray cone angle are classed as being compliant, and sprays having a cone angle that is less than said predetermined spray cone angle are classed as being non-compliant.

FIGS. 4 to 6 show images obtained with the method and the device of the invention, and in which it is possible to measure and/or to evaluate the cone angle of the spray, as shown by the cone lines.

FIGS. 7 and 8 show images respectively showing a test-fluid spray that is classed as being compliant (FIG. 7) and a test-fluid spray that is classed as being non-compliant (FIG. 8).

The present invention presents numerous advantages, and in particular:
it enables the spray from various types of spray device to be inspected automatically;
it enables said spray devices to be analyzed non-destructively;
it uses a setup that is compact and that can easily be adapted;
it uses components that are simple and standard, and thus generally inexpensive;
it enables image processing to be robust, and that may be performed in real time; and
it guarantees good repeatability and good discrimination between compliant and non-compliant sprays.

The present invention is described above with reference to various advantageous embodiments, but naturally any useful modification could be applied thereto by the person skilled in the art, without going beyond the ambit of the present invention, as defined by the accompanying claims.

1. A method of analyzing a spray generated by a spray device for spraying pharmaceutical fluid, the method being characterized in that it comprises the following steps:
providing a spray head (1) of a spray device for spraying pharmaceutical fluid, said spray head (1) including a spray orifice (2);
causing a test fluid to pass through said spray head (1) towards said spray orifice (2), said test fluid being air at a temperature that is different from ambient temperature;
displaying, by strioscopy, the flow of test fluid leaving said spray orifice (2); and
analyzing said display of the test-fluid flow so as to determine whether or not the test-fluid spray coming from said spray head complies with predetermined specifications.

2. A method according to claim 1, wherein said analyzing step includes determining the cone angle of the test-fluid spray.

3. A method according to claim 1, wherein said analyzing step includes an image-processing step for processing said display of the test-fluid flow.

4. A method according to claim 1, wherein said predetermined specifications include a predetermined spray cone angle, such that test-fluid sprays having a cone angle that is greater than or equal to said predetermined spray cone angle are classed as being compliant, and test-fluid sprays having a cone angle that is less than said predetermined spray cone angle are classed as being non-compliant.

5. A device for analyzing a spray generated by a spray device for spraying pharmaceutical fluid, the device being characterized in that it comprises:
a spray head (1) of a spray device for spraying pharmaceutical fluid, said spray head (1) including a spray orifice (2);
means (24) for causing a test fluid to pass through said spray head (1) towards said spray orifice (2), said test fluid being air at a temperature that is different from ambient temperature;
a strioscopic setup (20) for displaying, by strioscopy, the flow (5) of test fluid leaving said spray orifice (2); and
analyzer means (30) for analyzing said display of the test-fluid flow (5) so as to determine whether or not the test-fluid spray coming from said spray head (1) complies with predetermined specifications.

6. A device according to claim 5, wherein said analyzer means (30) include measuring means for measuring the cone angle of the test-fluid spray.

7. A device according to claim 5, wherein said analyzer means (30) include image-processing means for processing said display of the test-fluid flow.

8. A device according to claim 5, wherein said strioscopic setup (20) comprises a camera (21), an objective lens (22), a light source (23), generator means (24) for generating a test-fluid flow, a display zone (25), at least one collimator lens (26, 27), and a filter (28).

9. A device according to claim 8, wherein said strioscopic setup (20) further comprises a concave and/or parabolic mirror (29).

10. A device according to claim 5, wherein said filter (28) is a point, a wire, or a blade.
